(19) United States
(12) Patent Application Publication
(10) Pub. No.: US 2021/0193170 A1
(43) Pub. Date: Jun. 24, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Ryota MIZUTANI, Kanagawa (JP); Akira ICHIBOSHI, Kanagawa (JP); Kazunari KOMATSUZAKI, Kanagawa (JP); Shingo UCHIHASHI, Kanagawa (JP)

(73) Assignee: FUJI XEROX CO., LTD., Tokyo (JP)

(21) Appl. No.: 16/888,977

(22) Filed: Jun. 1, 2020

(30) Foreign Application Priority Data
Dec. 23, 2019 (JP) 2019-231255

(51) Int. Cl. G10L 25/63; A61B 5/00; A61B 5/16; G16H 15/00; G16H 50/50

(52) U.S. Cl. CPC G10L 25/63; A61B 5/4803; G06K 9/00335; G16H 15/00; G16H 50/50; A61B 5/165

(57) ABSTRACT

An information processing apparatus includes a processor. The processor is configured to perform control for outputting an evaluation result indicating whether a quality of communication performed between users is good or poor. The evaluation result is obtained by evaluating the quality of the communication based on information indicating a type of a scene where the communication is performed and information indicating a state of the communication identified in accordance with biologically-related information acquired from the users.

### CROSS-REFERENCE TO RELATED APPLICATIONS

This application is based on and claims priority under 35 USC 119 from Japanese Patent Application No. 2019-231255 filed Dec. 23, 2019.

### BACKGROUND

### (i) Technical Field

The present disclosure relates to information processing apparatuses and non-transitory computer readable media.

### (ii) Related Art

Japanese Unexamined Patent Application Publication No. 2019-101928 proposes an example of an information processing apparatus that controls the creativity of communication.

The information processing apparatus described in Japanese Unexamined Patent Application Publication No. 2019-101928 includes a calculating unit, a designing unit, and a presenting unit. The calculating unit calculates the activity of the autonomic nervous system of each participant belonging to a scene of a group by using biological information measured by a measuring device that measures the biological information of the participant. The designing unit designs a progress plan of communication in the scene of the group in accordance with a design mode corresponding to the calculated activity. The presenting unit presents the designed progress plan.

### SUMMARY

Aspects of non-limiting embodiments of the present disclosure relate to an information processing apparatus and a non-transitory computer readable medium that are capable of assisting with improvements in communication performed in business operations.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including a processor. The processor is configured to perform control for outputting an evaluation result indicating whether a quality of communication performed between users is good or poor. The evaluation result is obtained by evaluating the quality of the communication based on information indicating a type of a scene where the communication is performed and information indicating a state of the communication identified in accordance with biologically-related information acquired from the users.

### BRIEF DESCRIPTION OF THE DRAWINGS

An exemplary embodiment of the present disclosure will be described in detail based on the following figures, wherein:

FIG. 1 schematically illustrates an example of the configuration of an information processing system according to an exemplary embodiment of the present disclosure;

FIG. 2 is a block diagram illustrating an example of a control system of an information processing apparatus shown in FIG. 1;

FIG. 3 illustrates an example of communication type information;

FIG. 4 illustrates another example of the communication type information;

FIG. 5 illustrates an example of communication state information;

FIG. 6 illustrates an example of a feedback information table;

FIG. 7 is a flowchart illustrating an example of operation performed by the information processing apparatus shown in FIG. 1;

FIG. 8 illustrates an example of the configuration of a behavior-conversation-information acquiring apparatus shown in FIG. 1;

FIGS. 9A to 9C illustrate an example of detection of a conversation by behavior-conversation-information acquiring apparatuses, FIG. 9A illustrating an example of activities of users in a meeting room, FIGS. 9B and 9C being timing charts illustrating examples of signals obtained from the behavior-conversation-information acquiring apparatuses;

FIG. 10 illustrates an example of the configuration of a biological-information acquiring apparatus shown in FIG. 1;

FIG. 11 is a block diagram illustrating an example of a control system of an information processing apparatus according to a modification;

FIG. 12 schematically illustrates an example of a variation in a communication quality in a team;

FIGS. 13A to 13C illustrate examples of a scene status; FIG. 13A illustrating an example of a good status, FIG. 13B illustrating an example of an intermediate status, FIG. 13C illustrating an example of a poor status; and

FIG. 14 is a table collectively illustrating a specific example of the behavior-conversation-information acquiring apparatus and the biological-information acquiring apparatus.

### DETAILED DESCRIPTION

An exemplary embodiment of the present disclosure will be described below with reference to the drawings. In the drawings, components substantially having identical functions are given the same reference signs, and redundant descriptions thereof are omitted.

### Exemplary Embodiment

Information Processing System 1

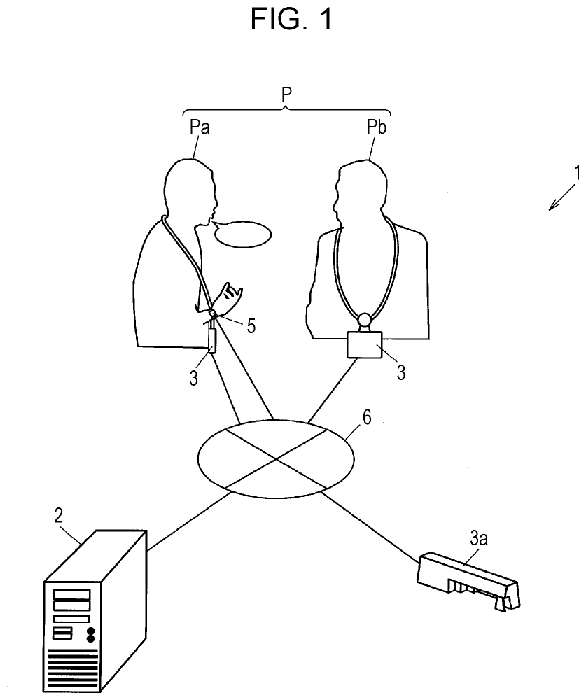

FIG. 1 illustrates an example of the configuration of an information processing system 1 according to an exemplary embodiment of the present disclosure. The information processing system 1 is applied to a place or an area (also referred to as “activity area” hereinafter) where users P are active. Examples of the activity area include a room (including a rental office and a shared office, and also referred to as “office” hereinafter), a workplace, such as a factory, and a learning place, such as a school or a classroom. FIG. 1 illustrates a case where the information processing system 1 is applied to an office.

As shown in FIG. 1, the information processing system 1 includes an information processing apparatus 2, a behavior-conversation-information acquiring apparatus 3, a base station 3a, a biological-information acquiring apparatus 5, and a network 6. The behavior-conversation-information acquiring apparatus 3 acquires behavior-related information (also referred to as “behavior data” hereinafter) and conversation-related information (also referred to as “conversation data” hereinafter) of users Pa and Pb. The behavior data and the conversation data may also be collectively referred to as “behavior conversation data” hereinafter. The behavior conversation data will be described in detail later. The base station 3a has a function of measuring the distance from the behavior-conversation-information acquiring apparatus 3 by using the radio field intensity. The biological-information acquiring apparatus 5 acquires biologically-related information (also referred to as “biological data” hereinafter) of each of the users Pa and Pb. The network 6 connects the behavior-conversation-information acquiring apparatus 3, the base station 3a, the biological-information acquiring apparatus 5 and the information processing apparatus 2 in a communicable manner.

The behavior-conversation-information acquiring apparatus 3 and the biological-information acquiring apparatus 5 may be worn by each of the users Pa and Pb or may be disposed distant from each of the users Pa and Pb. The base station 3a is fixedly provided at a predetermined position. Of the users Pa and Pb, the one speaking may be referred to as “speaker Pa”, the other may be referred to as “listener Pb”, and both of them may be collectively referred to as “users P”, “participants P” or “members P” if the speaker Pa and the listener Pb are not to be distinguished from each other. Each of the components will be described in detail below.

### Information Processing Apparatus 2

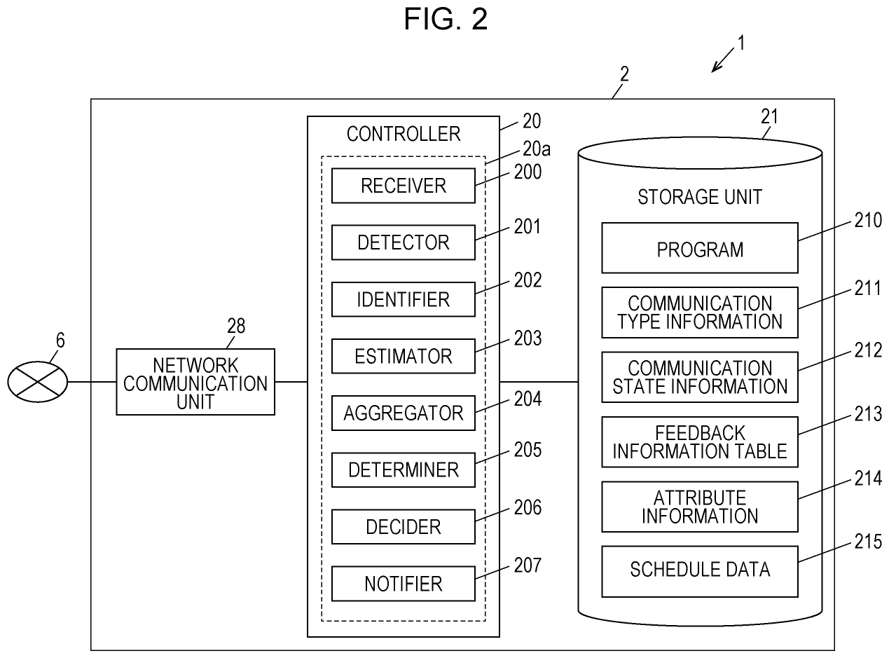

FIG. 2 is a block diagram illustrating an example of a control system of the information processing apparatus 2. The information processing apparatus 2 is a server apparatus constituted of, for example, a personal computer. The information processing apparatus 2 includes a controller 20 that controls each component, a storage unit 21 that stores various types of data, and a network communication unit 28 that communicates with an external apparatus (such as the behavior-conversation-information acquiring apparatus 3, the base station 3a, the biological-information acquiring apparatus 5, and a terminal apparatus) via the network 6.

Controller 20

The controller 20 is constituted of, for example, a processor 20a, such as a central processing unit (CPU), and an interface. The processor 20a operates in accordance with a program 210 stored in the storage unit 21 so as to function as, for example, a receiver 200, a detector 201, an identifier 202, an estimator 203, an aggregator 204, a determiner 205, a decider 206, and a notifier 207. The components 200 to 207 will be described in detail later.

Storage Unit 21

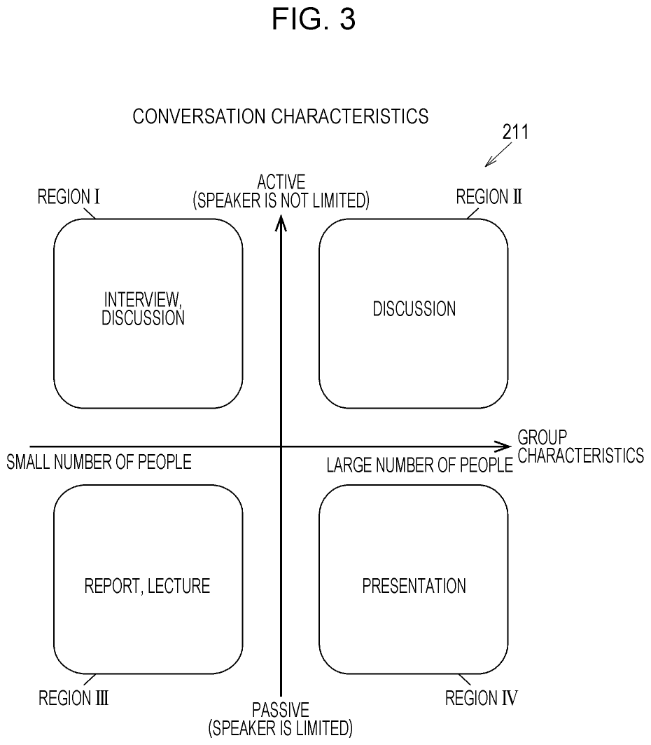

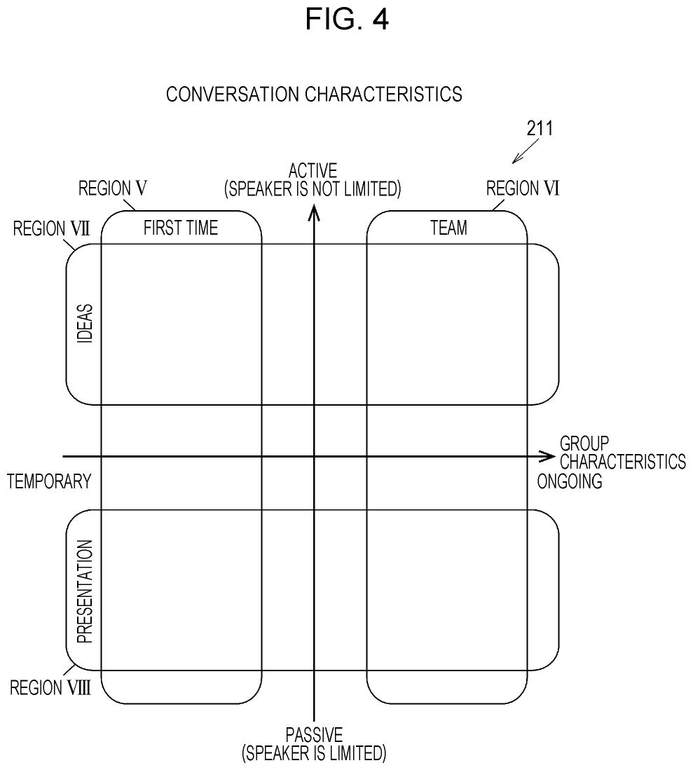

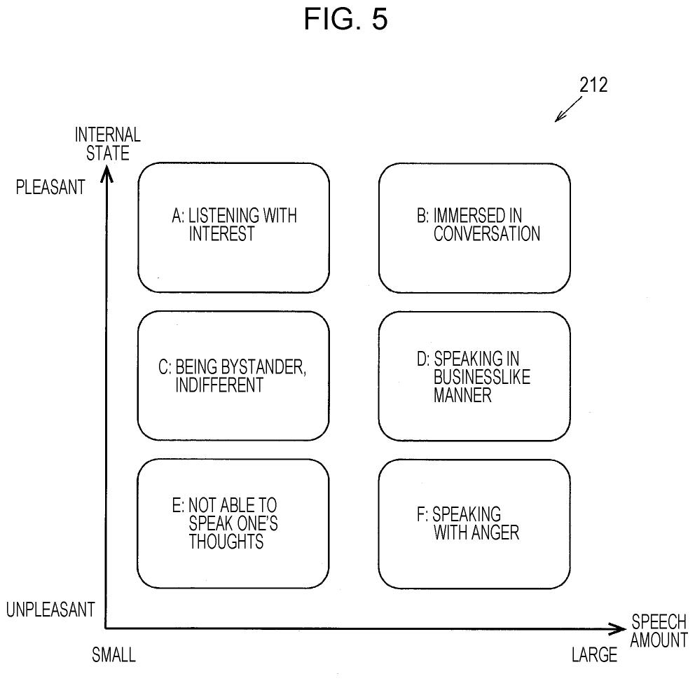

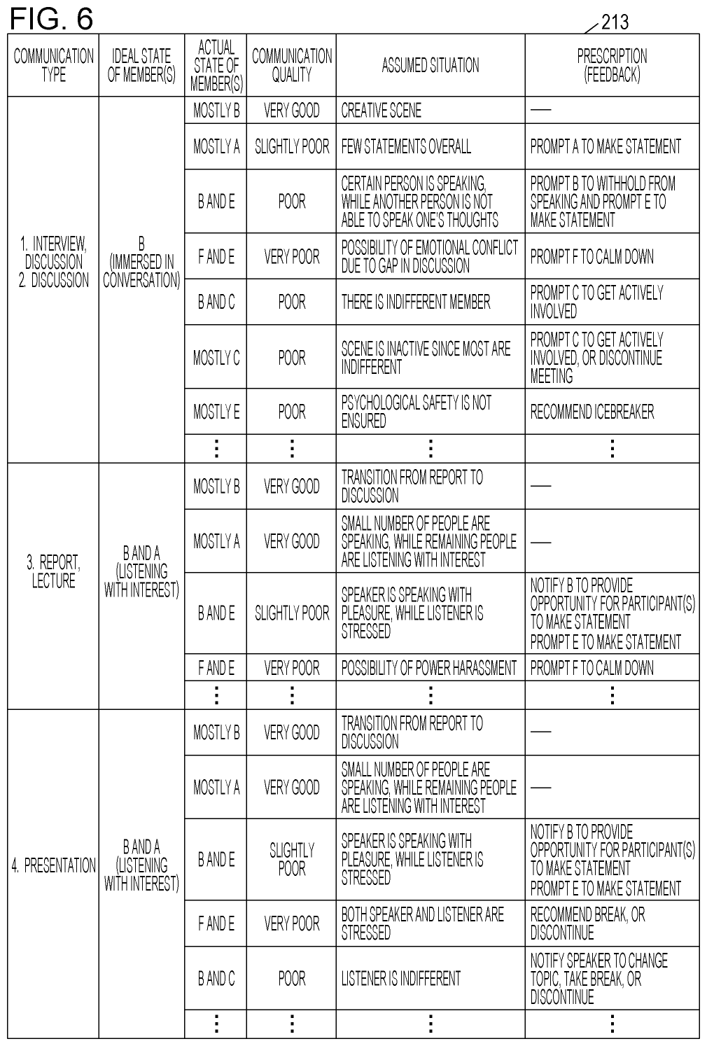

The storage unit 21 is constituted of, for example, a read-only memory (ROM), a random access memory (RAM), and a hard disk, and stores therein various types of data, such as the program 210, communication type information 211 (see FIGS. 3 and 4), communication state information 212 (see FIG. 5), a feedback information table 213 (see FIG. 6), attribute information 214, and schedule data 215.

The attribute information 214 indicates the attributes of each user P, such as the name, division, business title, social status, rank, and years of experience. The schedule data 215 indicates what kind of schedule each user P may have in a certain period. The communication type information 211, the communication state information 212, and the feedback information table 213 will be described in detail later.

Network Communication Unit 28

The network communication unit 28 is realized by, for example, a network interface card (NIC), and exchanges information and signals with external apparatuses via the network 6.

Components of Controller 20

Receiver 200

The receiver 200 receives, for example, various types of data, information, and signals transmitted from an external apparatus. In detail, the receiver 200 receives behavior conversation data transmitted from the behavior-conversation-information acquiring apparatus 3. Moreover, the receiver 200 receives biological data transmitted from the biological-information acquiring apparatus 5.

Detector 201

The detector 201 detects a specific signal from the various types of data received by the receiver 200. For example, the detector 201 detects a signal indicating a speech from the behavior conversation data. Moreover, for example, the detector 201 also detects information related to the detected speech, such as information for identifying the speaker Pa, information for specifying the position of the speaker Pa, and information indicating whether or not a conversation with another participant P is being made.

Identifier 202

In accordance with the behavior conversation data received by the receiver 200, the identifier 202 identifies the type (sometimes simply referred to as “type” or “communication type” hereinafter) of a scene where the users P are communicating with each other.

In detail, the identifier 202 checks the behavior conversation data against the communication type information 211 stored in the storage unit 21, so as to identify which region classified in the communication type information 211 the behavior conversation data corresponds to.

Examples of the communication type include a type classified in accordance with the characteristics of the communication, such as the scale and mode of the communication, and a type classified in accordance with the situation, such as the purpose, intention, and content of the communication, and the characteristics of the participants. The type classified in accordance with the characteristics of the communication includes, for example, “interview and discussion”, “discussion”, “report (or lecture)”, and “presentation”. The type classified in accordance with the situation includes, for example, “a situation where many participants are meeting for the first time”, “brainstorming of ideas”, and “team meeting”.

Examples of data used for identifying the communication type include the length of a speech by a participant P and the number of times and the frequency of a speech (also referred to as “speech amount” hereinafter), the evenness (also referred to as “balance” hereinafter) in the speech amount if there are multiple participants P, information derivable from the behavior conversation data, such as the number of participants P, the attribute information 214 of each participant P, and pre-recorded information, such as the schedule data 215 indicating the schedule of each participant P. The information derivable from the behavior conversation data may be calculated by the identifier 202 from the behavior conversation data.

Estimator 203

The estimator 203 estimates how each participant P is feeling about the communication, that is, the internal state (also referred to as “communication state” hereinafter) that each participant P has with respect to the communication.

In detail, the estimator 203 estimates the communication state by checking internal information (to be described later) of each participant P obtained from the behavior conversation data and the biological data against the communication state information 212 stored in the storage unit 21 and by identifying which region classified in the communication state information 212 the behavior conversation data and the biological data correspond to.

The communication state is expressed with items including an expression indicating a subjective view, such as how a user P feels about the communication, and an expression indicating an action taken by the user P. In detail, the communication state is expressed with items, such as “listening with interest”, “immersed in conversation”, and “speaking with anger” (see FIG. 5).

The internal state of a user P includes, for example, the mental state, the psychological state, and the emotional state of the user P. Examples of the internal state of a user P include “pleasantness/unpleasantness” indicating whether the user P tends to be in a pleasant state or tends to be an unpleasant state, “stress” indicating a psychological load on the user P, and “emotion” indicating the emotions of the user P.

The “pleasantness/unpleasantness”, “stress”, and “emotion” expressing the internal state of each user P may be evaluated by using a quantitative indicator. This indicator is obtained by analyzing the biological data of each user P. This analysis may be performed by the estimator 203.

Aggregator 204

The aggregator 204 aggregates communication states. In detail, the aggregator 204 aggregates the communication state estimated for each user P by the estimator 203, so as to determine the communication state in the group where the communication is carried out.

For example, the aggregator 204 performs an aggregation for determining what proportion of members P in a specific communication state is occupying the group or for determining whether a member P having any of the communication states is mixed in the group. The “proportion” may be qualitative information, such as “mostly A”.

Determiner 205

The determiner 205 determines whether the communication is good or poor (also referred to as “communication quality” hereinafter) in accordance with the communication type and the communication state. The criterion for determining whether the communication is “good” or “poor” may be set in advance.

In detail, the determiner 205 checks the communication type identified by the identifier 202 and the communication state estimated by the estimator 203 against the feedback information table 213 stored in the storage unit 21, so as to extract the corresponding quality, thereby determining the communication quality.

Moreover, the determiner 205 determines whether or not a prescription (also referred to as “feedback” hereinafter) is necessary in accordance with the communication type and the communication state.

Decider 206

If the determiner 205 determines that feedback is necessary, the decider 206 decides on the contents and method of the feedback in accordance with the communication type and the communication state.

In detail, the decider 206 checks the communication type identified by the identifier 202 and the communication state estimated by the estimator 203 against the feedback information table 213 stored in the storage unit 21, thereby deciding on the corresponding contents and method of the feedback.

Notifier 207

The notifier 207 performs the feedback in accordance with the decision made by the decider 206.

Information and Table Stored in Storage Unit 21

Communication Type Information 211

FIG. 3 illustrates an example of the communication type information 211. The communication type information 211 is used for identifying the communication type from behavior conversation data. In detail, in the communication type information 211, at least one type of information (i.e., parameter) for identifying the communication type and a numerical range corresponding to the parameter are recorded in the form of, for example, a table. For illustrative purposes, each of FIGS. 3 and 4 is a visualized map showing two types of parameters for identifying the communication type in a plane constituted of two orthogonal coordinate axes.

As shown in FIG. 3, communication types are classified in accordance with, for example, the group characteristics and the conversation characteristics during communication. The group characteristics correspond to, for example, the number of members P forming the group (i.e., participants P of the communication). The conversation characteristics correspond to, for example, an indicator indicating whether the conversation is dynamic or static, that is, whether the speaker Pa is limited to a specific participant P.

In detail, in a case where the group characteristics correspond to a small number of people and the conversation characteristics correspond to a dynamic conversation (region I), such a case corresponds to “interview and discussion” as a communication mode in which the speaker changes frequently between a small number of people. In a case where the group characteristics correspond to a large number of people and the conversation characteristics correspond to a dynamic conversation (region II), such a case corresponds to “discussion” as a communication mode in which the speaker changes frequently among a large number of people.

In a case where the group characteristics correspond to a small number of people and the conversation characteristics correspond to a static conversation (region III), such a case corresponds to “report” or “lecture” as a communication mode in which a specific participant P tends to be speaking between a small number of people. In a case where the group characteristics correspond to a large number of people and the conversation characteristics correspond to a static conversation (region IV), such a case corresponds to “presentation” as a communication mode in which a specific participant P tends to be speaking among a large number of people.

FIG. 4 illustrates another example of the communication type information 211. As shown in FIG. 4, communication types may be classified by using continuity as the group characteristics indicating whether the group is temporary or ongoing.

In detail, a case where the group is temporary (region V) corresponds to communication in a situation where many participants P are meeting for the first time. A case where the group is ongoing (region VI) corresponds to communication in a situation with certain collectivity, as in a team meeting.

A case of dynamic conversation characteristics (region VII) corresponds to communication intended for giving out ideas among participants P, as in brainstorming. A case of static conversation characteristics (region VIII) corresponds to communication such as a presentation.

Sections where the aforementioned regions V to VIII overlap correspond to communication having the characteristics of the corresponding regions. For example, a case of temporary group characteristics and dynamic conversation characteristics (V and VI) corresponds to communication in a situation where many participants P are meeting for the first time and are brainstorming for giving out ideas. Detailed descriptions for combinations other than the combination of V and VI will be omitted.

Communication State Information 212

FIG. 5 illustrates an example of the communication state information 212. The communication state information 212 is used for identifying the communication state for each member P. In detail, in the communication state information 212, at least one type of parameter for identifying the communication state and a numerical range corresponding to the parameter are recorded in the form of, for example, a table. Similar to FIGS. 3 and 4, for illustrative purposes, FIG. 5 is a visualized map showing two types of parameters for identifying the communication state in a plane constituted of two orthogonal coordinate axes.

As shown in FIG. 5, communication states are classified in accordance with, for example, a speech amount of each participant P and an evaluation value indicating “pleasant” or “unpleasant” as an example of an indicator indicating the internal state of the participant P. Furthermore, examples of the classified communication states include six states, namely, “A: listening with interest”, “B: immersed in conversation”, “C: being bystander, indifferent”, “D: speaking in a businesslike manner”, “E: not able to speak one's thoughts”, and “F: speaking with anger”. The number and the contents of the classified items are not limited to the above.

In detail, the communication state is classified as a “listening” state or a “speaking” state as an action in accordance with the speech amount, and is classified as an active state with an interested or immersed mindset or as a passive state with an oppressed, pressured, tolerating, or angry mindset, in accordance with the internal state.

In more detail, for example, if the speech amount tends to be small and the internal state tends to be “unpleasant”, the communication state is classified as a state where a participant P is oppressed from speaking due to certain pressure and is listening one-sidedly, that is, the state of “E: not able to speak one's thoughts”. As another example, if the speech amount tends to be large and the internal state tends to be “pleasant”, the communication state is classified as the state of “B: immersed in conversation”.

Feedback Information Table 213

FIG. 6 illustrates an example of the feedback information table 213. The feedback information table 213 is used for determining the communication quality and for identifying the contents and method of feedback by pairing the communication type and the communication state.

The feedback information table 213 is provided with a “communication type” field, an “ideal state of member(s)” field, an “actual state of member(s)” field, a “communication quality” field, an “assumed situation” field, and a “prescription (feedback)” field. Among these fields, the “communication type” field and the “actual state of member(s)” field have input values therein, whereas the “communication quality” field and the “prescription (feedback)” field have output values therein in accordance with the input values. Reference signs “A” to “F” indicated in the fields respectively correspond to “A” to “F” defined in the communication state information 212 shown in FIG. 5.

In the “communication type” field, the communication types mentioned above are recorded. FIG. 6 exemplarily lists only the regions I to IV described with reference to FIG. 3.

In the “ideal state of member(s)” field, a predetermined ideal communication state is recorded for each communication type. For example, if the communication type is “interview” or “discussion”, the state of “B: immersed in conversation” shown in FIG. 5 is defined as being an ideal state.

In the “actual state of member(s)” field, the communication state of the members P is recorded. Examples of information recorded in the “actual state of member(s)” field include “mostly B” (i.e., the communication state of most members P among the members P forming the group is the state of “B: immersed in conversation”), “mostly A” (i.e., the communication state of most members P among the members P forming the group is the state of “A: listening with interest”), and “F and E” (i.e., there is a mixture of members P in the state of “E: not able to speak one's thoughts” and members P in the state of “F: speaking with anger” in the group). These pieces of information are checked against information obtained by the aggregator 204 qualitatively aggregating, for each group, the communication state estimated for each member P by the estimator 203.

In the “communication quality” field, information indicating the communication quality is recorded. Examples of the information indicating the communication quality include “very good”, “slightly poor”, “poor”, and “very poor”.

The communication quality does not necessarily have to be classified into four levels as in the above example, and may be classified into two levels or three levels, or may be classified in more detail into five or more levels. Alternatively, the communication quality may be expressed quantitatively by using a numerical value.

Information recorded in the “assumed situation” field indicates the type of situation occurring in the communication and assumed when the communication state is the state recorded in the “actual state of member(s)” field.

Information recorded in the “prescription (feedback)” field indicates the contents and method of feedback to be performed in accordance with the communication quality. Examples of the information recorded in the “prescription (feedback)” field include “prompt A to make statement” (i.e., prompt a member P in the state of “A: listening with interest” among the members P forming the group to make a statement) and “prompt F to calm down” (i.e., prompt a member P in the state of “F: speaking with anger” among the members P forming the group to calm down). In the table, reference symbol “-” indicates that feedback is not particularly necessary.

### Operation According to Exemplary Embodiment

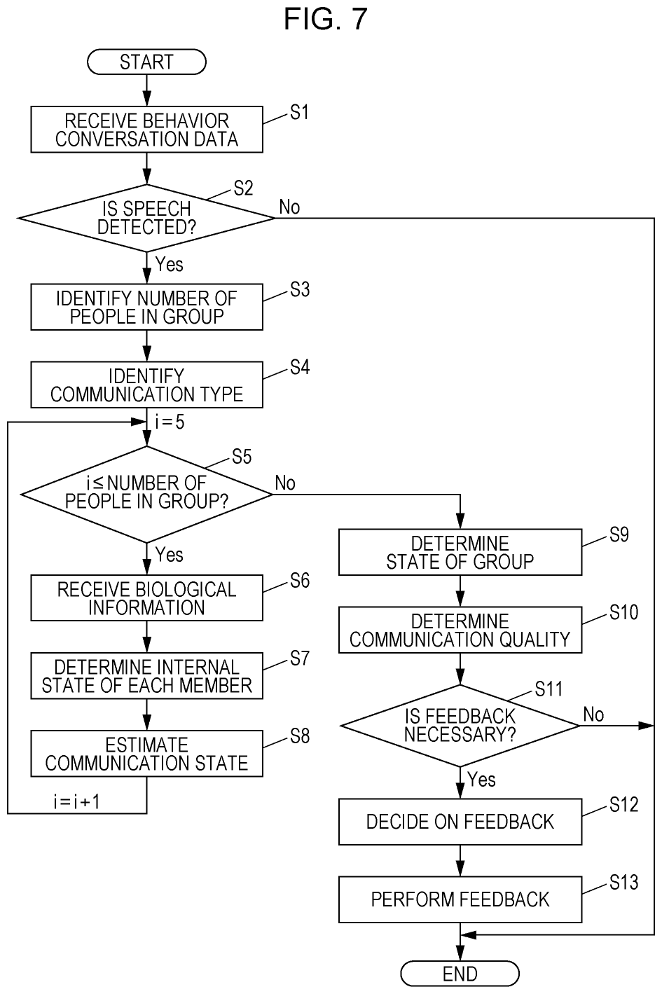

FIG. 7 is a flowchart illustrating an example of the operation of the information processing apparatus 2 according to the exemplary embodiment of the present disclosure. In step S1, the receiver 200 receives behavior conversation data acquired by the behavior-conversation-information acquiring apparatus 3 and transmitted to the information processing apparatus 2. In step S2, the detector 201 detects a signal indicating a speech from the behavior conversation data.

When the detector 201 detects a speech from the behavior conversation data (YES in step S2), the identifier 202 identifies a group formed by members P who are speaking, and identifies the number of members P forming the group (sometimes simply referred to as “number of people in the group” hereinafter) in step S3.

Then, in step S4, the identifier 202 identifies the communication type. In this case, the identifier 202 may refer to the attribute information 214 and the schedule data 215 stored in advance in the storage unit 21.

In step S6, the receiver 200 receives biological information, acquired by the biological-information acquiring apparatus 5 and transmitted to the information processing apparatus 2, related to each of the members P forming the group. The estimator 203 determines the internal state of each member P in accordance with, for example, the biological information in step S7, and estimates the communication state of each member P in accordance with, for example, the internal state in step S8.

The process from step S6 to step S8 involving the reception of the biological information by the receiver 200 and the determination of the internal state and the estimation of the communication state by the estimator 203 is performed on all of the members P in the group (YES in step S5).

Subsequently, in step S9, the aggregator 204 aggregates the communication state of each member P so as to determine the communication state of the group. In step S10, the determiner 205 refers to the feedback information table 213 so as to determine the communication quality according to the communication type and the communication state.

In step S11, the determiner 205 further determines whether or not feedback is necessary by referring to the feedback information table 213. If the determiner 205 determines that feedback is necessary (YES in step S11), the decider 206 refers to the feedback information table 213 so as to decide on the contents and method of feedback in step S12.

In step S13, the notifier 207 performs feedback in accordance with the decision by the decider 206.

Behavior-Conversation-Information Acquiring Apparatus 3

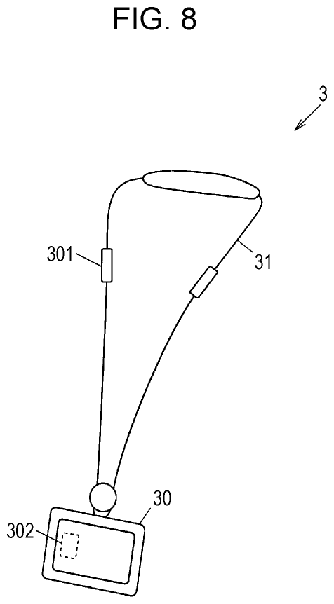

FIG. 8 illustrates an example of the configuration of the behavior-conversation-information acquiring apparatus 3. The behavior-conversation-information acquiring apparatus 3 acquires information related to a behavior and a speech or conversation.

As shown in FIG. 8, for example, the behavior-conversation-information acquiring apparatus 3 includes a base unit 30 equipped with a sensor that acquires conversation data, and also includes a strap 31 for securely retaining the base unit 30 to a position close to the body of a user P. For example, the behavior-conversation-information acquiring apparatus 3 is used by being hung from the neck of the user P.

The base unit 30 includes multiple microphones 301 and 302 that are disposed at different distances from the mouth of the user P in a state where the strap 31 is hung from the neck of the user P. In detail, the multiple microphones 301 and 302 include a first microphone 301 provided on the strap 31 and a second microphone 302 provided in the base unit 30.

Accordingly, the multiple microphones 301 and 302 are provided at different distances from the mouth of the user P in this manner. Thus, when a voice output by the user P is detected, a time lag occurs in the speech detection timings, whereas when a voice output by a third person is detected, such a time lag in the speech detection timings is minimized. The behavior-conversation-information acquiring apparatus 3 utilizes this principle to distinguish and identify the voices of first and third persons from each other.

Furthermore, the behavior-conversation-information acquiring apparatus 3 measures the distance between multiple base stations 3a so as to identify the position and the behavior of the user P.

In this exemplary embodiment, the behavior-conversation-information acquiring apparatus 3 may be of any type that is capable of acquiring information about the position and the speech of the user P, and may be, for example, a detector that contains a camera and a directional microphone.

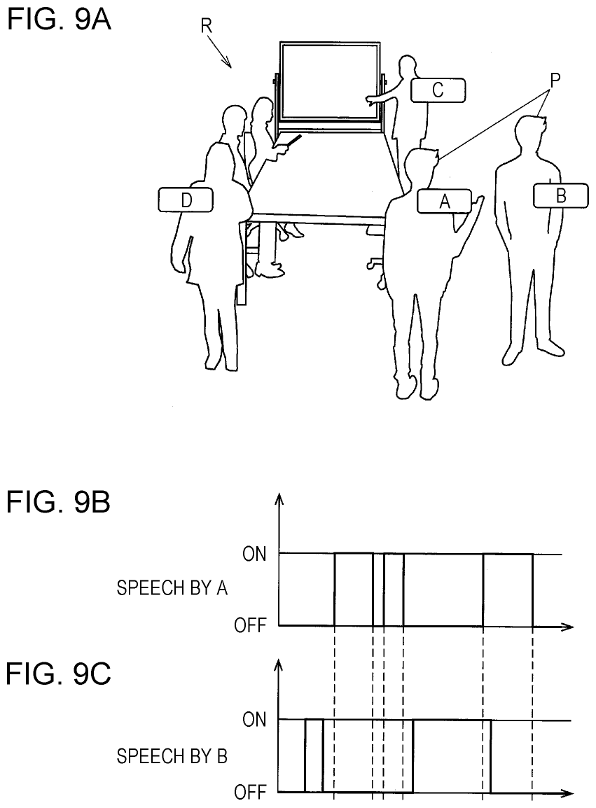

FIGS. 9A to 9C illustrate an example of detection of a conversation by behavior-conversation-information acquiring apparatuses 3. Specifically, FIG. 9A illustrates an example of activities of users P in a meeting room, and FIGS. 9B and 9C are timing charts illustrating examples of signals obtained from the behavior-conversation-information acquiring apparatuses 3. Although the actual users P are individually wearing the behavior-conversation-information acquiring apparatuses 3, the behavior-conversation-information acquiring apparatuses 3 are not shown in FIG. 9A.

As shown in FIG. 9A, it is assumed that multiple (e.g., six) users P are active in the activity area, such as a meeting room R. It is assumed that two of the multiple users P (i.e., “A” and “B”) are having a conversation.

In this case, the behavior-conversation-information acquiring apparatuses 3 acquire signals as shown in FIGS. 9B and 9C. With regard to the ordinate axis, “ON” denotes a state where a user is speaking, whereas “OFF” denotes a state where the user is not speaking. For example, in a case where two different people are conversing with each other, the signals obtained each indicate that the speaking state alternately appears, as shown in FIGS. 9B and 9C.

Biological-Information Acquiring Apparatus 5

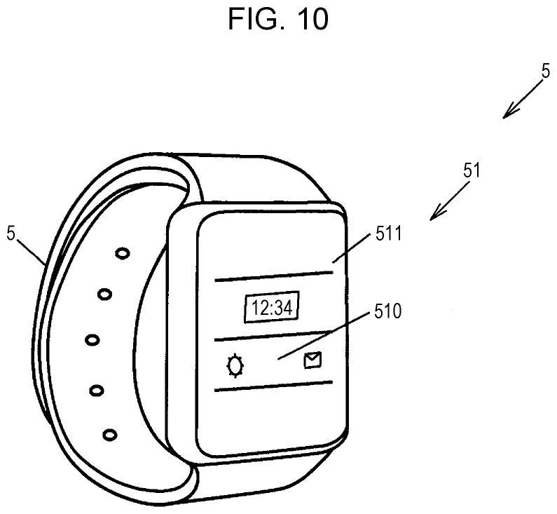

FIG. 10 illustrates an example of the configuration of the biological-information acquiring apparatus 5. For example, the biological-information acquiring apparatus 5 measures biological data of a user P in the activity area when the user P is active. The biological-information acquiring apparatus 5 may measure the biological data not only when the user P is active but also when, for example, the user P is in an inactive state, such as when the user P is lying down, napping, or sleeping.

The biological data is released from a biological body and may include any of the following examples:

a. information indicating a body motion (e.g., acceleration caused by a body motion, a pattern indicating a behavior, and so on);

b. an amount of activity (e.g., the number of steps taken, consumed calories, and so on); and

c. vital information (e.g., the heart rate, the pulse wave, the pulse rate, the respiration rate, the body temperature, the blood pressure, and so on).

In this exemplary embodiment, the biological-information acquiring apparatus 5 particularly measures, for example, data related to the balance of the autonomic nervous system, such as a heartbeat interval (e.g., seconds or milliseconds), a low-frequency component (LF), and a high-frequency component (HF).

The biological-information acquiring apparatus 5 is desirably of a wearable type worn on the body of the user P. In this exemplary embodiment, the biological-information acquiring apparatus 5 is of a wristband type worn on a wrist, as shown in FIG. 10. The biological-information acquiring apparatus 5 includes a base unit 51 and a wearable unit 52. The base unit 51 includes a sensor unit 510 equipped with various types of sensors and also includes an operation display 511 used for displaying measured data, inputting information, and performing operations. The wearable unit 52 is formed of a belt to be worn on the wrist. The sensor unit 510 acquires various types of biological data from the body, which is in contact with the base unit 51, at a predetermined timing or cycle. The sensor unit 510 includes, for example, an accelerometer that measures a body motion.

The biological-information acquiring apparatus 5 is not limited to a wristband type and may be of any type capable of acquiring biological data. Examples of the biological-information acquiring apparatus 5 include a ring type worn on a finger, a belt type worn on the waist, a shirt type that is worn on the upper body and comes into contact with, for example, the left and right arms, the shoulders, the chest, and the back, a head type that covers the head, an eyeglasses type or a goggle type worn on the head, an earphone type worn on an ear, and an attachable type attached to a part of the body. Furthermore, the biological-information acquiring apparatus 5 does not necessarily have to be worn on the body and may be, for example, a camera having a function for measuring the heart rate by capturing the absorption of light by hemoglobin.

Network 6

The network 6 is a communication network, such as a local area network (LAN), a wide area network (WAN), the Internet, or an intranet, and may be a wired network or a wireless network.

First Modification

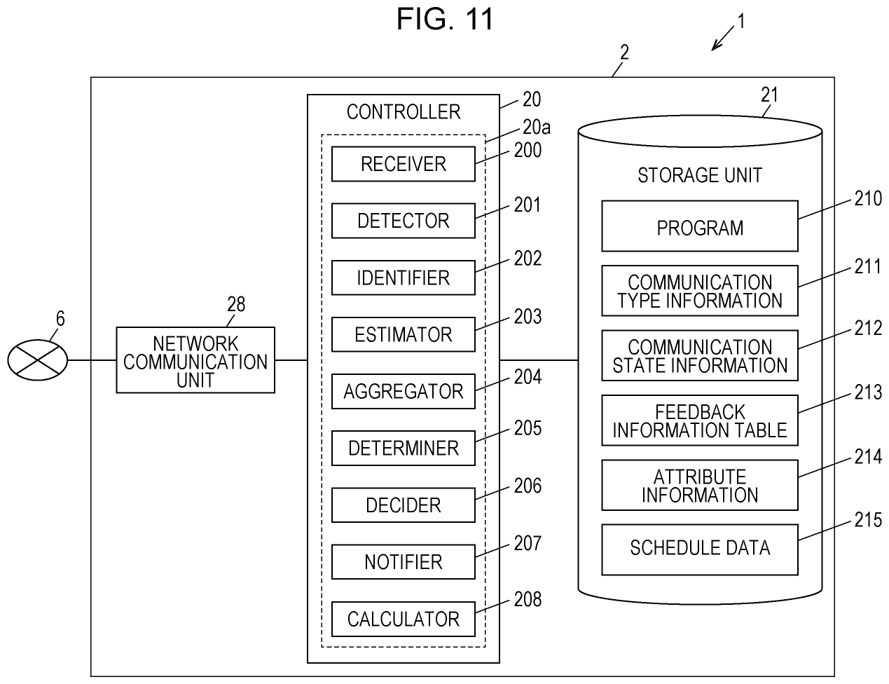

FIG. 11 is a block diagram illustrating an example of a control system of an information processing apparatus 2 according to a first modification. The information processing apparatus 2 according to this modification is different from the information processing apparatus 2 according to the above exemplary embodiment in that the information processing apparatus 2 further includes a calculator 208. Components having configurations and functions identical to those in the above exemplary embodiment are given the same reference signs, and detailed descriptions thereof will be omitted. Moreover, the following description focuses on differences from the information processing apparatus 2 according to the above exemplary embodiment.

As shown in FIG. 11, the processor 20a in the information processing apparatus 2 according to this modification further functions as the calculator 208, in addition to the components 200 to 207 described in the above exemplary embodiment.

The calculator 208 aggregates the communication quality in accordance with a predetermined calculation method (i.e., an algorithm) for each team, so as to calculate an index (also referred to as “communication index” or “team communication quality index (TCQI)”) for comprehensively determining the communication state of the team.

Furthermore, the calculator 208 further analyzes a tendency (also referred to as “trend” hereinafter) of a temporal variation in the TCQI, and outputs the communication state of the team in a visualized form. Moreover, when the TCQI crosses a predetermined threshold value, the calculator 208 outputs a warning indicating that the communication state has deteriorated.

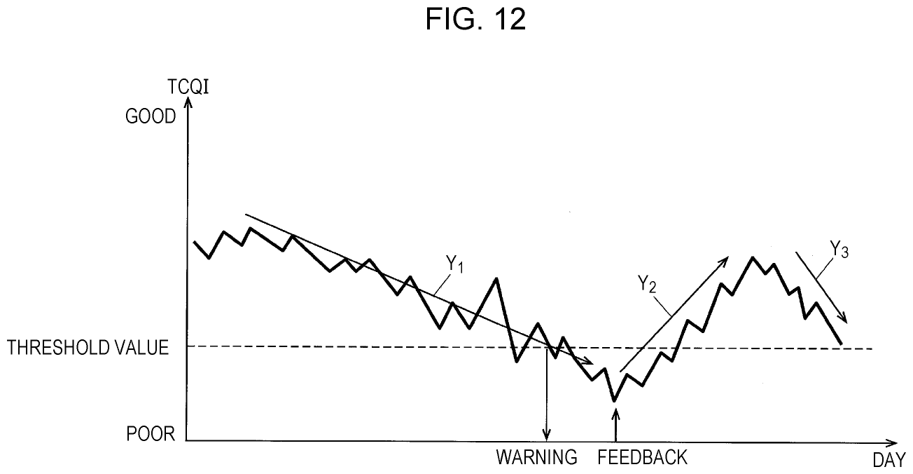

FIG. 12 schematically illustrates an example of a variation in the communication quality in a team. The ordinate axis denotes the TCQI. In this case, a larger value is defined as “good”, whereas a smaller value is defined as “poor”. The abscissa axis denotes a time axis indicated in units of, for example, “days”.

As shown in FIG. 12, when the communication state in the team gradually deteriorates, the TCQI decreases with time (see an arrow “Y₁”). When this TCQI falls below the predetermined threshold value, the calculator 208 outputs a warning.

Assuming that certain feedback is performed after the warning is output and the communication state in the team is improved, the TCQI increases again (see an arrow “Y₂”). The TCQI subsequently continues to increase for a certain period, and then may tend to decrease again (see an arrow “Y₃”). In such a case, when the TCQI falls below the predetermined threshold value again, the calculator 208 outputs a warning again. The feedback in this case is not necessarily limited to the contents recorded on the feedback information table 213 shown in FIG. 6. For example, a principal member of the team may be prompted to have a one-on-one conversation with another member or to provide an opportunity to have a conversation within the team.

Second Modification

With regard to the communication state of a team, for example, an amount S indicating a “scene status” evaluated based on distribution of amounts of speech and levels of stress (also referred to as “stress levels” hereinafter) may be used as an indicator. A stress indicator may be determined from biological data of a speaker Pa. The stress indicator used here is a value obtained by dividing a low-frequency component (LF) of a heart beat by a high-frequency component (HF). Stress is an example of the internal state of the speaker Pa.

The amount S indicating the scene status may be determined by using, for example, Expression (1) indicated below:

amount S indicating scene status=VAR (speech amount)×AR (stress/speech amount) (1)

where “VAR” is a function expressing the degree of distribution and is used for calculating an evaluation value. With regard to the “VAR”, it is assumed that the evaluation value is output in three levels, namely, large, medium, and small, with respect to the speech amount, and is output in three levels, namely, high, medium, and low, with respect to “stress/speech amount”.

With regard to the value of S, the smaller the value, the better the scene status, and the larger the value, the poorer the scene status. The “stress/speech amount” is a value obtained by normalizing the stress indicator based on the speech amount, and may be, for example, a value obtained by dividing the stress indicator by the speech amount. The “stress/speech amount” is an example of a stress level.

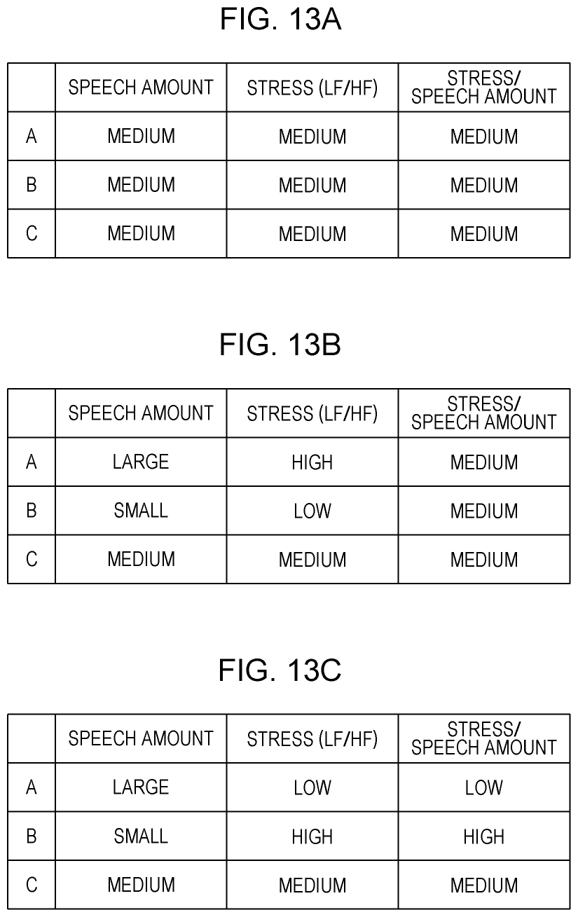

FIGS. 13A to 13C illustrate examples of the scene status (S). Specifically, FIG. 13A illustrates an example of a good status, FIG. 13B illustrates an example of an intermediate status, and FIG. 13C illustrates an example of a poor status. As shown in FIG. 13A, in a case where the evenness in the speech amounts and the evenness in the stress levels among the participants P are both high, the value of S is small, that is, indicates a good status.

As shown in FIG. 13B, in a case where the evenness in the speech amounts among the participants P is low but the stress levels are about the same, the value of S is an intermediate value, that is, indicates an intermediate status.

As shown in FIG. 13C, in a case where the evenness in the speech amounts and the evenness in the stress levels among the participants P are both low, the value of S is large, that is, indicates a poor status.

Third Modification

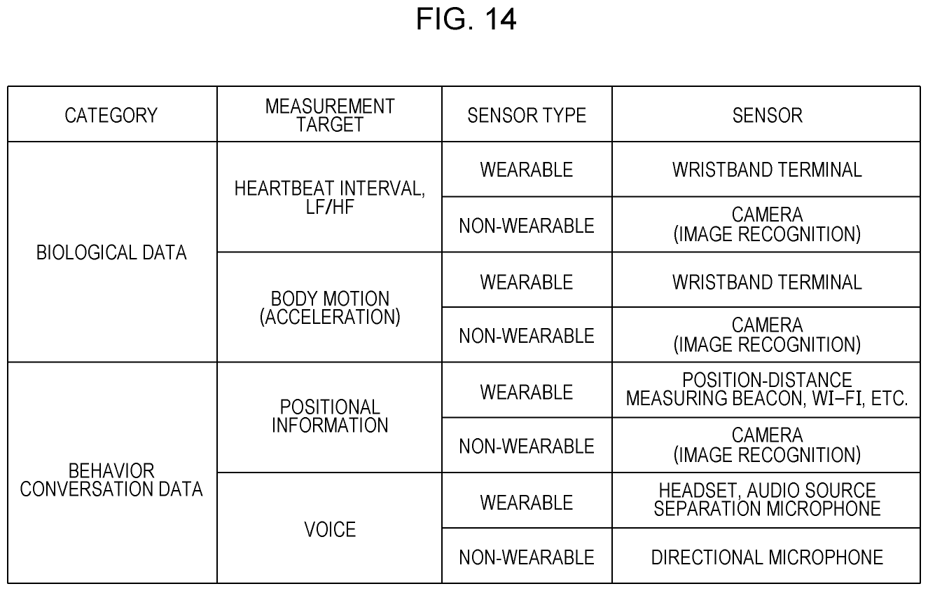

FIG. 14 is a table collectively illustrating a specific example of the behavior-conversation-information acquiring apparatus 3 and the biological-information acquiring apparatus 5. The behavior-conversation-information acquiring apparatus 3 and the biological-information acquiring apparatus 5 are not limited to those described in the above exemplary embodiment. As shown in FIG. 14, the behavior-conversation-information acquiring apparatus 3 used may be of a non-wearable type, such as a camera having an image recognition function.

Although the exemplary embodiment of the present disclosure has been described above, the exemplary embodiment of the present disclosure is not limited to the above exemplary embodiment, and various modifications are permissible so long as they do not depart from the scope of the disclosure. For example, the variations of “communication type” are not limited to those mentioned above. For example, the communication type may be identified by using only a single type of parameter (e.g., only the number of people or the conversation characteristics) instead of using two types of parameters. Moreover, the “communication type” is not necessarily limited to information identified by the identifier 202 and may alternatively be manually-input information.

Each component of the controller 20 may partially or entirely be constituted of a hardware circuit, such as a Field Programmable Gate Array (FPGA) or an Application Integrated Circuit (ASIC).

Furthermore, one or some of the components in the above exemplary embodiment may be omitted or changed. Moreover, in the flowchart in the above exemplary embodiment, for example, a step or steps may be added, deleted, changed, or interchanged within the scope of the disclosure. The program used in the above exemplary embodiment may be provided by being recorded on a computer readable recording medium, such as a compact disc read-only memory (CD-ROM). Alternatively, the program used in the above exemplary embodiment may be stored in an external server, such as a cloud server, and may be used via a network.

In the exemplary embodiment above, the term “processor” refers to hardware in a broad sense. Examples of the processor include general processors (e.g., CPU: Central Processing Unit) and dedicated processors (e.g., GPU: Graphics Processing Unit, ASIC: Application Integrated Circuit, FPGA: Field Programmable Gate Array, and programmable logic device).

In the exemplary embodiment above, the term “processor” is broad enough to encompass one processor or plural processors in collaboration which are located physically apart from each other but may work cooperatively. The order of operations of the processor is not limited to one described in the exemplary embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The exemplary embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to
perform control for outputting an evaluation result indicating whether a quality of communication performed between users is good or poor, the evaluation result being obtained by evaluating the quality of the communication based on information indicating a type of a scene where the communication is performed and information indicating a state of the communication identified in accordance with biologically-related information acquired from the users.

2. The information processing apparatus according to claim 1, wherein the processor performs control for identifying the type of the scene in accordance with a characteristic of a group formed by the users and a characteristic of a conversation performed in the communication.

3. The information processing apparatus according to claim 2,
wherein the processor performs the control for identifying the type of the scene in accordance with a number of the users or continuity of the conversation in the group as the characteristic of the group.

4. The information processing apparatus according to claim 1,
wherein the processor performs control for estimating the quality of the communication in accordance with information indicating an amount of a speech by each user and internal information obtained from the biologically-related information and indicating an internal state of each user with respect to the communication.

5. The information processing apparatus according to claim 2,
wherein the processor performs control for estimating the quality of the communication in accordance with information indicating an amount of a speech by each user and internal information obtained from the biologically-related information and indicating an internal state of each user with respect to the communication.

6. The information processing apparatus according to claim 3,
wherein the processor performs control for estimating the quality of the communication in accordance with information indicating an amount of a speech by each user and internal information obtained from the biologically-related information and indicating an internal state of each user with respect to the communication.

7. The information processing apparatus according to claim 4,
wherein the processor performs the control for estimating the quality of the communication by using an indicator indicating a trend of a psychologically pleasant state or a trend of a psychologically unpleasant state as the internal information.

8. The information processing apparatus according to claim 5,
wherein the processor performs the control for estimating the quality of the communication by using an indicator indicating a trend of a psychologically pleasant state or a trend of a psychologically unpleasant state as the internal information.

9. The information processing apparatus according to claim 6,
wherein the processor performs the control for estimating the quality of the communication by using an indicator indicating a trend of a psychologically pleasant state or a trend of a psychologically unpleasant state as the internal information.

10. An information processing apparatus comprising:
a processor configured to
perform control for outputting information indicating a countermeasure for improving communication performed between users, the information being obtained from information indicating a type of a scene where the communication is performed and information indicating a state of the communication identified in accordance with biologically-related information of the users.

11. A non-transitory computer readable medium storing a program causing a computer to execute a process, the process comprising:
outputting an evaluation result indicating whether a quality of communication performed between users is good or poor, the evaluation result being obtained by evaluating the quality of the communication based on information indicating a type of a scene where the communication is performed and information indicating a state of the communication identified in accordance with biologically-related information acquired from the users.
